(19) United States
(12) Patent Application Publication
(10) Pub. No.: US 2010/0145206 A1
(43) Pub. Date: Jun. 10, 2010

(54) ALTERNANS AND CARDIAC ISCHEMIA

(75) Inventors: Lahn Fendelander, Arlington, MA (US); Ali Haghighi-Mood, Andover, MA (US); Richard J. Cohen, Chestnut Hill, MA (US)

Correspondence Address: FISH & RICHARDSON P.C., P.O. BOX 1022, MINNEAPOLIS, MN, 55440-1022, US

(73) Assignee: Cambridge Heart, Inc., Bedford, MA (US)

(21) Appl. No.: 12/630,735

(22) Filed: Dec. 3, 2009

Related U.S. Application Data
(60) Provisional application No. 61/120,148, filed on Dec. 5, 2008.

(51) Int. Cl. A61B 5/0402; A61B 5/02

(52) U.S. Cl. 600/513; 600/517

(57) ABSTRACT

One or more electrocardiographic signals are detected from a subject. The occurrence of alternans in the electrocardiographic signals are detected using one or more processors. One or more characteristics of detected alternans are determined. The determined characteristics of the detected alternans are analyzed to determine whether cardiac ischemia is present.

### CROSS-REFERENCE TO RELATED APPLICATIONS

This application claims priority to U.S. Provisional Application No. 61/120,148, filed on Dec. 5, 2008 and titled “ALTERNANS AND ISCHEMIA-RELATED CORONARY ARTERY DISEASE,” which is incorporated by reference in its entirety.

### TECHNICAL FIELD

This disclosure is directed to the measurement of alternans in conjunction with testing for coronary artery disease and cardiac ischemia.

### BACKGROUND

The coronary arteries are the blood vessels responsible for delivering blood to the heart muscle (the myocardium). Coronary artery disease (“CAD”) involves the deposit over time of atherosclerotic plaque on the internal walls of these arteries. The plaque deposits can restrict the flow of blood and thereby prevent the artery from delivering an adequate amount of oxygenated blood to the myocardium. Tissue which receives an inadequate amount of oxygenated blood is termed “ischemic.” Coronary artery disease thus may lead to ischemia of the heart muscle (“cardiac ischemia” or “myocardial ischemia”). Coronary artery disease may be insufficient to cause cardiac ischemia when the patient is at rest, but cardiac ischemia may develop during physiologic stress, such as exercise, when myocardial demand for oxygen is increased.

CAD may develop for decades without the patient experiencing any physical symptoms. Therefore, the patient may be unaware of significant risk of myocardial infarction, sudden cardiac death (SCD), and heart failure. The atherosclerotic plaque deposits can spontaneously rupture and create a blockage, leading to acute myocardial infarction (death of the muscle tissue supplied by the coronary artery). Acute myocardial infarction can cause death by means of pump failure or electrical instability. Regions of cardiac tissue which are periodically ischemic due to the presence of the CAD may become electrically unstable while they are ischemic leading to SCD. Heart muscle which is chronically starved of oxygen may become altered in its physical structure and weaken. This condition is known as “cardiomyopathy” and can lead to the heart being less capable of pumping blood efficiently (“heart failure”). Cardiomyopathy also predisposes to electrical instability which may lead to SCD.

### SUMMARY

In general, in some aspects, a method for detecting cardiac ischemia includes receiving one or more electrocardiographic signals from a subject and detecting, using at least one processor, the occurrence of alternans in the electrocardiographic signals. The method also includes determining one or more characteristics of detected alternans and analyzing the determined characteristics of the detected alternans to determine whether cardiac ischemia is present.

This and other implementations can optionally include one or more of the following features, which also may optionally be in any combination. For example, determining the characteristics of the detected alternans can include determining the location of detected alternans within an electrocardiogram waveform. Analyzing the determined characteristics of the detected alternans can include analyzing the determined location of the detected alternans to determine whether the cardiac ischemia is present. Determining the characteristics of the detected alternans can include evaluating a relationship of the detected occurrence of the alternans to cardiac stress and analyzing the determined characteristics of the detected alternans can include analyzing the evaluated relationship to provide an indication of whether the subject has cardiac ischemia.

Also, receiving one or more electrocardiographic signals from the subject can include receiving one or more electrocardiographic signals from the subject while the subject is undergoing a stress test. The stress can be exercise stress, pharmacological stress, or stress induced by electrically pacing the heart. Determining the characteristics of the detected alternans can include determining an onset heart rate of alternans or a maximum heart rate below which alternans is not present. The method can also include determining the occurrence, in the electrocardiographic signals, of abnormalities that persist over multiple beats and are indicative of cardiac ischemia and providing an indication of whether the subject has cardiac ischemia based on the determined characteristics of the alternans and the determination of the occurrence of abnormalities that persist over multiple beats and are indicative of cardiac ischemia. The abnormalities can be alterations in the ST segment. The alterations in the ST segment can be depression or elevation of the ST segment or a change in the slope of the ST segment.

Further, the electrocardiographic signals can be received using an ambulatory electrocardiography device. The alternans can be detected using a spectral method of analysis. The alternans can be detected using an analytic method of analysis. The method may additionally include obtaining non-electrocardiographic measures indicative of the presence of cardiac ischemia and providing an indication of whether the subject has cardiac ischemia based on the obtained non-electrocardiographic measures indicative of the presence of cardiac ischemia. The non-electrocardiographic measures can be measured using echocardiographic imaging of a heart or by characterizing the uptake of radionuclides by the heart.

Moreover, determining one or more characteristics of the detected alternans can include determining a power or magnitude of alternans. Detecting the occurrence of alternans in the electrocardiographic signals can include detecting the occurrence of T-wave alternans occurring in the electrocardiographic signals. Ddetecting the occurrence of alternans in the electrocardiographic signals can include detecting the occurrence of QRS complex alternans occurring in the electrocardiographic signals. Detecting the occurrence of alternans in the electrocardiographic signals can include detecting the occurrence of ST segment alternans occurring in the electrocardiographic signals. The method can further include generating cardiac signal data from the electrocardiographic signals and segmenting the cardiac signal data into cardiac signal data segments which include cardiac signal data of sequential heart beats. At least one cardiac signal data segment can partially overlap the cardiac signal data of at least one other cardiac signal data segment. Finally, the method can include sorting the cardiac signal data segments.

In other implementations, some aspects include a computer-readable medium encoded with a computer program comprising instructions that, when executed, operate to cause one or more computers to perform operations. The operations include receiving cardiac signal data generated from measured heart beats of a subject and detecting the occurrence of alternans in the cardiac signal data. The operations also include determining one or more characteristics of detected alternans in the cardiac signal data and providing an indication related to cardiac ischemia based on the determined characteristics of detected alternans.

This and other implementations can optionally include one or more of the following features, which also may optionally be in any combination. For example, determining the characteristics of the detected alternans can include determining the location of detected alternans within an electrocardiogram waveform and providing the indication related to cardiac ischemia based on the detected occurrence of alternans can include providing the indication related to cardiac ischemia based on the determined location of detected alternans. Detecting the occurrence of alternans in the cardiac signal data can include detecting the occurrence of T-wave alternans in the cardiac signal data. Detecting the occurrence of alternans in the cardiac signal data can include detecting the occurrence of QRS complex alternans in the cardiac signal data. Detecting the occurrence of alternans in the cardiac signal data can include detecting the occurrence of ST segment alternans in the cardiac signal data.

Also, receiving the cardiac signal data can include accessing stored cardiac signal data from a non-volatile data storage, where the cardiac signal data was stored by an ambulatory electrocardiography device. Receiving the cardiac signal data can include accessing cardiac signal data from volatile memory which has not been stored in a non-volatile data storage. Determining the characteristics of the detected alternans can include determining an onset heart rate of alternans or a maximum heart rate below which alternans is not present in the cardiac signal data.

Further, the operations can also include determining the occurrence, in the cardiac signal data, of abnormalities that persist over multiple beats and are indicative of cardiac ischemia. Providing the indication related to cardiac ischemia based on the determined characteristics of detected alternans can include providing the indication of whether the subject has cardiac ischemia based on the determined characteristics of detected alternans and the determination of the occurrence of abnormalities that persist over multiple beats and are indicative of cardiac ischemia. The abnormalities can be alterations in the ST segment. The alterations in the ST segment can be depression or elevation of the ST segment or a change in the slope of the ST segment.

In other implementations, some aspects include a system, the system includes sensors configured to measure electrical activity of heart beats, an amplifier configured to amplify the electrical activity, and an analog to digital converter configured to convert the electrical activity to cardiac signal data. The system also includes a processor configured to receive the cardiac signal data generated from measured heart beats of a subject and detect the occurrence of alternans in the cardiac signal data. The processor is also configured to determine one or more characteristics of detected alternans in the cardiac signal data and provide an indication related to cardiac ischemia based on the determined characteristics of detected alternans.

This and other implementations can optionally include one or more of the following features, which also may optionally be in any combination. For example, to determine the characteristics of the detected alternans, the processor can be configured to determine the location of detected alternans within an electrocardiogram waveform and to provide the indication related to cardiac ischemia based on the detected occurrence of alternans, the processor can be configured to provide the indication related to cardiac ischemia based on the determined location of detected alternans. To detect, the occurrence of alternans in the cardiac signal data, the processor can be configured to detect the occurrence of T-wave alternans in the cardiac signal data. To detect, the occurrence of alternans in the cardiac signal data, the processor can be configured to detect, the occurrence of QRS complex alternans in the cardiac signal data.

Also, to detect, the occurrence of alternans in the cardiac signal data, the processor can be configured to detect, the occurrence of ST segment alternans in the cardiac signal data. To determine the characteristics of the detected alternans, the processor can be configured to determine an onset heart rate of alternans or a maximum heart rate below which alternans is not present in the cardiac signal data. The processor can be configured to determine the occurrence, in the cardiac signal data, of abnormalities that persist over multiple beats and are indicative of cardiac ischemia and to provide the indication related to cardiac ischemia based on the determined characteristics of detected alternans, the processor can be configured to provide the indication of whether the subject has cardiac ischemia based on the determined characteristics of detected alternans and the determination of the occurrence of abnormalities that persist over multiple beats and are indicative of cardiac ischemia.

In other implementations, some aspects include a method for detecting cardiac ischemia, the method includes receiving first cardiac signal data generated from measured heart beats of a subject and determining characteristics of alternans occurring in the first cardiac signal data. The method also includes receiving second cardiac signal data generated from measured heart beats of the subject after the subject has undergone a change relating to cardiac stress and determining characteristics of alternans occurring in the second cardiac signal data. The method further includes analyzing a difference between the characteristics of alternans occurring in the first cardiac signal data and the characteristics of alternans occurring in the second cardiac signal data and providing an indication related to cardiac ischemia based on the analyzed difference between the characteristics of alternans.

This and other implementations can optionally include one or more of the following features, which also may optionally be in any combination. For example, determining characteristics of alternans occurring in the first cardiac signal data can include determining the location of alternans occurring in the first cardiac signal data, determining characteristics of alternans occurring in the second cardiac signal data can include determining the location of alternans occurring in the second cardiac signal data, analyzing a difference between the characteristics can include analyzing a difference between the location of alternans occurring in the first cardiac signal data and the location of alternans occurring in the second cardiac signal data, and providing the indication related to cardiac ischemia based on the analyzed difference between the characteristics of alternans can include providing the indication related to cardiac ischemia based on the analyzed difference between the location of alternans occurring in the first cardiac signal data and the location of alternans occurring in the second cardiac signal data.

Also, determining characteristics of alternans occurring in the first cardiac signal data can include determining a power or magnitude of alternans in the first cardiac signal data, determining characteristics of alternans occurring in the second cardiac signal data can include determining a power or magnitude of alternans in the second cardiac signal data, analyzing the difference can include determining the difference between the power or magnitude of alternans in the first cardiac signal data and the power or magnitude of alternans in the second cardiac signal data, and providing the indication can include assessing whether the difference between the power or magnitude of alternans is indicative of cardiac ischemia.

Further, determining characteristics of alternans occurring in the first cardiac signal data can include determining an onset heart rate of or a maximum heart rate without alternans in the first cardiac signal data, determining characteristics of alternans occurring in the second cardiac signal data can include determining an onset heart rate of or a maximum heart rate without alternans in the second cardiac signal data, analyzing the difference can include determining the difference between the onset heart rate of or the maximum heart rate without alternans in the first cardiac signal data and the onset heart rate of or the maximum heart rate without alternans in the second cardiac signal data, and providing the indication can include assessing whether the difference between the onset heart rate of or the maximum heart rate without alternans is indicative of cardiac ischemia. Receiving the first and second cardiac signal data can include accessing stored cardiac signal data from a non-volatile data storage, where the cardiac signal data was stored by an ambulatory electrocardiography device.

Moreover, the method can include determining the occurrence, in the first cardiac signal data, of abnormalities that persist over multiple beats and are indicative of cardiac ischemia, determining the occurrence, in the second cardiac signal data, of abnormalities that persist over multiple beats and are indicative of cardiac ischemia, and analyzing a difference between the occurrence of abnormalities in the first cardiac signal data and the occurrence of abnormalities in the second cardiac signal data. Providing the indication related to cardiac ischemia based on the analyzed difference between the characteristics of alternans can include providing the indication related to cardiac ischemia based on the analyzed difference between the characteristics of alternans and based on the analyzed difference between the occurrence of abnormalities.

In additional, the method can include segmenting the first cardiac signal data into first cardiac signal data segments, each first cardiac signal data segment including cardiac signal data of sequential heart beats, and segmenting the second cardiac signal data into second cardiac signal data segments, each second cardiac signal data segment including cardiac signal data of sequential heart beats. Determining characteristics of alternans occurring in the first cardiac signal data can include determining characteristics of alternans occurring in the first cardiac signal data segments, determining characteristics of alternans occurring in the second cardiac signal data can include determining characteristics of alternans occurring in the second cardiac signal data segments, and analyzing the difference can include analyzing the difference between the characteristics of alternans occurring in the first cardiac signal data segments and the characteristics of alternans occurring in the second cardiac signal data segments.

Finally, the first and second cardiac signal data can be segmented such that the sequential order of the heart beats as measured by sensors is maintained within the first and second cardiac signal data segments. The first and second cardiac signal data can be segmented such that the cardiac signal data in at least one cardiac signal data segment partially overlaps the cardiac signal data of another cardiac signal data segment.

The details of one or more implementations are set forth in the accompanying drawings and the description below. Other features will be apparent from the description and drawings, and from the claims.

### DESCRIPTION OF DRAWINGS

FIG. 1 is an example of a waveform of a heart beat measured by an electrocardiography device to produce cardiac signal data.

FIG. 2 is an illustration of a patient undergoing testing for ischemia due to CAD using an electrocardiography device to measure alternans.

FIG. 3 is a schematic of a electrocardiography device to measure alternans in testing for ischemia due to CAD.

FIG. 4A is a block diagram of a process to detect ischemia due to CAD by analyzing the location of alternans within the ECG waveform.

FIG. 4B is a block diagram of a process to detect ischemia due to CAD by analyzing characteristics of alternans before and after cardiac exertion.

FIG. 5 is a block diagram of a process to detect ischemia due to CAD using an electrocardiography device.

FIG. 6 is a diagram of a heart rate profile of cardiac signal data stored by an electrocardiography device.

FIG. 7 is a diagram of a heart rate profile of segmented cardiac signal data generated from the cardiac signal data of FIG. 6.

FIG. 8 is a diagram of sorted cardiac signal data generated from the segmented cardiac signal data of FIG. 7.

FIG. 9 is a schematic of a computer system configured to carry out the processes of FIGS. 4A-5.

FIG. 10 is an example of a waveform and an illustration of the relationship between action potential duration alternans, T-wave alternans, and the development of re-entrant arrhythmias.

### DETAILED DESCRIPTION

It is important to be able to non-invasively detect the presence of clinically significant coronary artery disease (“CAD”) at an early stage and before the patient suffers complications. Clinically significant CAD can be detected clinically by stressing the heart (for example by means of exercise stress) and then using non-invasive means to detect the development of cardiac ischemia. A coronary artery that is partially blocked may provide an adequate amount of blood flow to the heart muscle when the heart is not being stressed, but may provide an inadequate amount of blood flow when the heart is stressed and the heart muscle requires additional blood flow to supply additional oxygen. If the partially blocked vessel cannot provide this additional blood flow, the heart muscle becomes ischemic.

Cardiac ischemia may be diagnosed non-invasively in the doctor's office by means of a stress test. Stress tests can involve exercise stress, but may also involve pharmacologic stress or stress induced by electrically pacing the patient's heart. The readout of such a stress test can include visible changes in the electrocardiogram recorded during and after stress, changes in the image of the beating heart as detected using ultrasound imaging (echocardiography), or changes in the regional uptake by the heart muscle of an injected radionuclide which is detected using an imaging gamma camera.

If cardiac ischemia due to the presence of CAD is detected non-invasively, the patient can then be taken to the cardiac catheterization lab where dye can be injected directly into the coronary arteries for precise x-ray imaging of the coronary arteries. This may lead to treatment, for example, by placement of one or more coronary artery stents which force the coronary arteries to stay open or in other cases by coronary artery bypass graft surgery.

Some techniques used to detect cardiac ischemia non-invasively (e.g. visible changes in the electrocardiogram, ultrasound imaging, or radionuclide imaging) have limited precision. As such, some techniques can produce false positive and false negative tests results. Cardiac testing discussed below can be used alone or in conjunction with other technologies to aid in diagnosis of cardiac ischemia due to coronary artery disease.

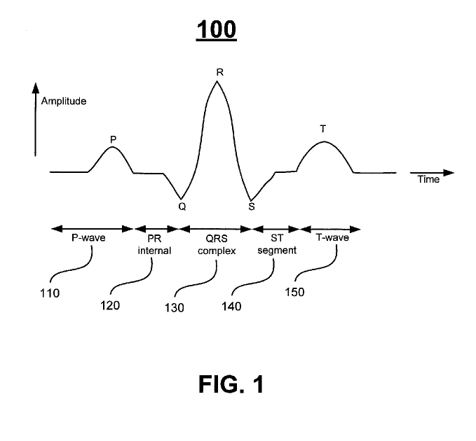

Patients can undergo cardiac testing to determine the presence of myocardial ischemia and risk of SCD. Cardiac testing can include measuring electrical characteristics of a heart beat. FIG. 1 is an example of a waveform 100 of a heart beat measured by an electrocardiography device to produce cardiac signal data. In particular, the waveform 100 is a measurement of a voltage between two electrodes placed on the body surface. The waveform 100 corresponds to a single heart beat. Various portions of the waveform 100 represent electrical activity in various structures of the heart. The P-wave 110 of the waveform 100 appears at initiation of the beat and corresponds to electrical activation of the atria of the heart. The PR interval 120 corresponds to the time between the end of the P-wave 110 and the onset of the QRS complex 130. There is normally no measurable electrical activity during the PR interval and this interval is often used to set the zero baseline of the recording. The QRS complex 130 corresponds to the electrical activation of the ventricles. The ST segment 140 represents the period between the end of the QRS complex and the onset of the T-wave 150, and corresponds to the portion of time during which the ventricles are activated (depolarized). In normal individuals, the ST segment tends to be relatively flat or slightly up-sloping and is approximately at the zero baseline. However, the ST segment can be shifted up or down or have a nonzero slope in patients with myocardial disease. The T-wave 150 reflects the electrical recovery of the ventricles.

The electrocardiogram measured at rest, during exercise and during recovery from exercise can be used to detect cardiac ischemia from CAD and electrical instability that leads to SCD. For example, in an exercise test, cardiac ischemia may be detected by identifying a downward shifting of the ST segment during recovery from exercise (“ST segment depression”).

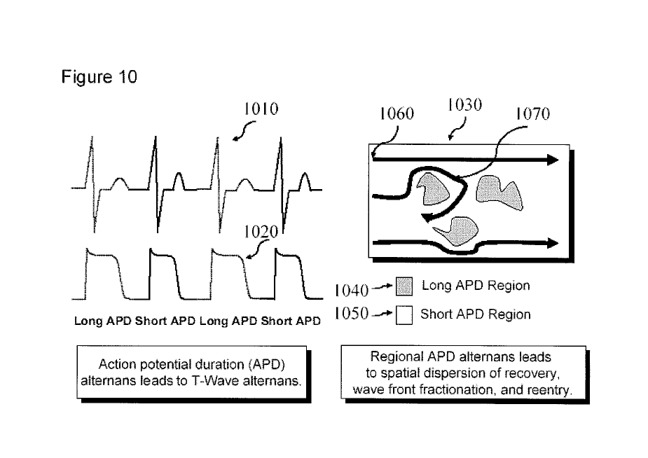

Alternans is a beat-to-beat pattern of variation of an electrocardiographic complex (specifically, an every-other-beat pattern of variation in shape or magnitude). T-wave alternans is a beat-to-beat pattern of variation in the T-wave 150 of the electrocardiographic complex (specifically, an every-other-beat pattern of variation in the shape of the T-wave 150). An example of T-wave alternans is shown in the first ECG measurement 1010 of FIG. 10. The presence of T-wave alternans can indicate electrical instability of the ventricles. Clinically significant T-wave alternans can reflect a variation in the shape of the T-wave of only a few microvolts. These tiny changes in the shape of the T-wave may not be able to be seen by means of visual inspection of the electrocardiogram. Reliable measurement of microvolt T-wave alternans can require specialized equipment incorporating high fidelity recording and sophisticated signal processing algorithms to be able to detect, for example, a very small every-other-beat pattern of variation in the presence of other temporal patterns of beat-to-beat variability in the waveform 100. The equipment may also need to be able to effectively reject noise and other artifacts. This presence of T-wave alternans can be used clinically as an indicator of increased risk of SCD from ventricular heart rhythm disturbances (“arrhythmias”).

Prolonged electrical recovery (“repolarization”) may occur regionally in diseased ventricular muscle tissue on an every-other-beat basis. This alternation in regional recovery causes a spatial inhomogeneity in electrical recovery processes across the ventricles which predisposes to the development of re-entrant ventricular arrhythmias such as ventricular tachycardia and ventricular fibrillation which cause sudden cardiac death. This phenomenon is illustrated in FIG. 10. The first ECG measurement 1010 demonstrates T-wave alternans in which the shape of the T-wave alternates on an every-other-beat basis.

The second ECG measurement 1020 represents the electrical activity of individual cardiac muscle cells which demonstrate action potential duration (APD) alternans—the action potential is the basic unit of electrical activity in individual cardiac muscle cells. The alternation in action potential duration in individual cells is manifested in the surface electrocardiogram as T-wave alternans.

The right hand panel 1030 depicts a section of ventricular myocardium. The right hand panel 1030 includes regional prolongation in recovery due to the regional prolongation of the action potentials on an every-other-beat basis. The gray areas 1040 demonstrate regions of ventricular muscle where, in a specific beat, the APD is long. The white areas 1050 demonstrate regions of ventricular muscle where, in a specific beat, the APD is short. In regions where the APD is short and electrical recovery has occurred, electrical activation wave fronts 1060 can propagate unimpeded. Electrical activation wave fronts 1070 cannot propagate through regions where the APD is prolonged and electrical recovery has not yet occurred. Hence these wave fronts 1070 fractionate and lead to the development of self-sustained re-entrant arrhythmias such as ventricular tachycardia and fibrillation. Thus, regional prolongation in recovery due to APD alternans leads to electrical wave front fractionation and the development of re-entrant ventricular arrhythmias that can lead to sudden cardiac death.

A wide range of diseases of the heart tissue can lead to the development of alternans and increased risk of SCD. For example, scar tissue resulting from prior myocardial infarction and cardiomyopathy due to long standing CAD can lead to the development of alternans and increased risk of SCD. Also, the disease known as “non-ischemic cardiomyopathy” also leads to the development of alternans and predisposes to SCD.

Ischemia due to the presence of CAD causes regional abnormalities in electrical processes in the affected myocardium. This can lead in turn to the development of alternans. Notably, however, the characteristics of alternans which result from ischemia may differ from alternans that results from other causes, such as cardiomyopathy. As such, the specific characteristics of alternans which result from ischemia can be considered as a mechanism to detect the presence of ischemia. In particular, partial blockage due to CAD may allow adequate oxygenated blood to flow during rest or early on in cardiac exertion (e.g., exercise) and, therefore, a patient suffering from CAD may only exhibit significant alternans during exercise or recovery from exercise. A patient at risk for SCD may have abnormalities of cardiac tissue present independent of cardiac exertion, leading to the occurrence of alternans at rest rather than only during or after exercise. Therefore, the context of the occurrence of a patient's alternans can be used to differentiate between alternans resulting from ischemia due to CAD and alternans due to other causes such as cardiomyopathy which predispose to SCD.

Also, the temporal distribution of alternans over a waveform 100 can differ based upon the cause of the alternans. One implementation utilizes the temporal distribution of alternans over a waveform to detect underlying ischemia. Ischemia can affect early repolarization of the heart muscles coinciding with the ST segment 140 and earlier part of the T-wave 150. Thus ischemia may be preferentially associated with alternans of the ST segment and of the early part of the T-wave. Even the QRS complex 130 may develop alternans. Alternans occurring later in the T-wave, coinciding with electrical recovery, can be indicative of cardiomyopathy or other chronic conditions. Therefore, the location of alternans within the waveform 100 can additionally be used to differentiate between alternans resulting from ischemia due to CAD and alternans resulting from other causes.

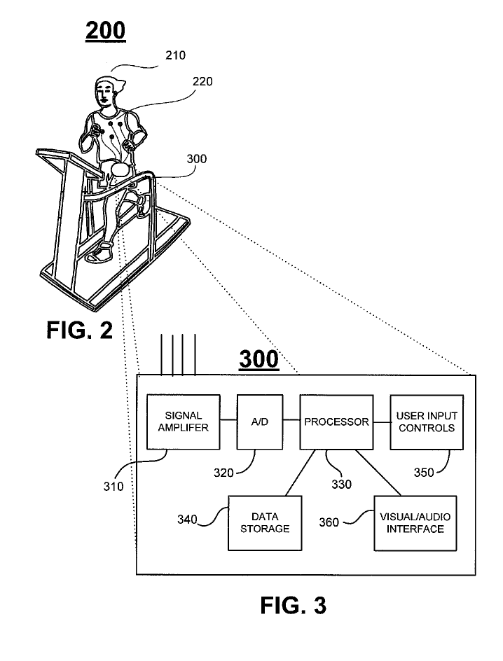

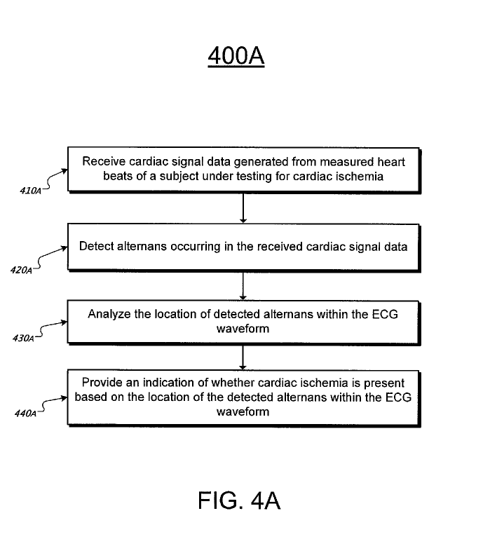

FIG. 2 is an illustration 200 of a patient 210 undergoing testing for ischemia due to coronary artery disease using an electrocardiography device 300 to measure alternans, and FIG. 3. is an exemplary schematic of the electrocardiography device 300. The cardiac signal data is processed with the electrocardiography device 300 to detect alternans indicative of ischemia due to CAD in the cardiac activity of the patient 210 before, during, or after cardiac exertion.

Alternans is generally measured as voltage changes as small as a few microvolts using an electrocardiogram (ECG) produced by the electrocardiography device 300. The ECG is a measurement of electrical activity of the heart. The waveform 100 represents the ECG corresponding to a single heart beat. The ECG can be recorded in a controlled setting, such as a hospital or doctor's office, to obtain cardiac signal data at a desired heart rate while controlling for noise. The presence and characteristics of alternans can depend upon heart rate, so testing can also include placing a patient on a treadmill to intentionally elevate the heart rate to create cardiac exertion. For example, an exercise tolerance test is a medical procedure where a patient is placed on a treadmill and monitored while the level of physical exertion is gradually increased. The monitoring can include generating an ECG with the electrocardiography device 300 to analyze changes in the characteristics of the waveform 100 during different levels of exercise.

An ambulatory electrocardiography device (AED) is a portable electrocardiography device 300 configured to be worn on a patient's person. The patient wears the AED outside of the hospital or doctor's office without having their mobility significantly limited. The AED measures and stores cardiac signals for an extended period of time (e.g., 24 hours). Also, AEDs often do not include an impedance measurement, which is generally included in electrocardiography devices to factor out noise. Consequently, the cardiac signal data produced by an AED can be of a wide range of heart rates and can have higher levels of noise. To compensate for these and/or other issues, the processing techniques used to analyze the AED's cardiac signal data to detect alternans can be different than those traditionally used to analyze the ECG of an electrocardiography device. For simplicity of understanding, the description below refers generally to an electrocardiography device 300 rather than an AED. Nevertheless, the description of the electrocardiography device 300 is also applicable to implementations using an AED.

Multiple electrodes 220 of the electrocardiography device 300 are attached to the chest of the patient 210 at particular locations of the patient's body to detect electrical activity from various sources. As shown, the electrocardiography device 300 includes a signal amplifier 310, an analog to digital converter 320, a processor 330, and data storage 340. The electrocardiography device 300 can also include user input controls 350 and a visual or audio interface 360. These features of the electrocardiography device 300 are exemplary; the electrocardiography device can include different or additional features.

An AED generally uses fewer electrodes 220 (e.g., three to eight) than an electrocardiography device 300 (e.g., ten) to enhance device mobility. An AED is generally worn at or around the waist to enable the patient 210 to walk and otherwise be mobile while the AED measures heart beats and records cardiac signals using the electrodes 220.

The signal amplifier 310 receives the cardiac signals measured from the electrodes 220 and amplifies them to produce amplified signal channels for processing. While an electrocardiography device 300 typically has about 12 channels, AEDs generally have less, such as three or four channels. The signal amplifier 310 can be an instrumentation amplifier or another differential amplifier.

The amplified channels of the cardiac signals are digitized by the analog to digital converter 320 and then sent to the processor 330. One or more of the measured signals may be used to determine and adjust for noise rather than to measure cardiac activity. For example, the electrocardiography device 300 may include a signal line to measure respiration and a signal line to measure impedance. These techniques are described in more detail in U.S. Pat. No. 5,713,367, entitled “Measuring and accessing cardiac electrical stability,” the contents of which are incorporated herein by reference.

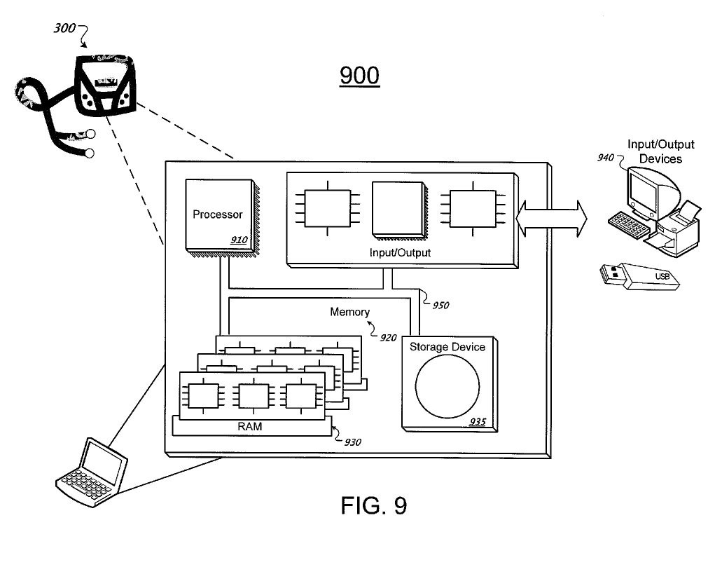

The data storage 340 can be a tangible computer-readable storage medium, such as, for example, a flash drive or a computer hard disk. The data storage 340 itself can be removable from the electrocardiography device 300 to enable uploading of the cardiac signal data to a computer or other device. The electrocardiography device 300 can include a data communication port (e.g., a universal serial bus or Ethernet interface) to interface with a separate computer to upload, display, or process the cardiac signal data. The transferability of the cardiac signal data in the data storage 340 is particularly useful in implementations using an AED to measure the cardiac signal data and the processor 330 to process the cardiac signal data with a separate device. Additional computer hardware and functionality which can be included in the electrocardiography device 300 is included in the description of FIG. 9.

The processor 330 can utilize the user input controls 350 and a visual or audio interface 360 to enable additional functionality to better enable the measurement of cardiac signals useful in detecting alternans. For example, alternans occurring as a result of ischemia due to CAD is more often detected at elevated heart rates (e.g., between 100 and 120 beats per minute). The user input controls 350 and the visual or audio interface 360 can be used to communicate whether additional signal data is needed from such an accelerated heart rate. The patient 210 can use this information to determine whether it is necessary to spend time under cardiac exertion to facilitate the desired measurement of cardiac signals.

Also, the processor 330 can use the visual or audio interface 360 in conjunction with the user input controls 350 to guide the administration of a programmed exercise tolerance test. For example, the electrocardiography device 300 can be used to instruct the beginning, elevation, and ending of cardiac exertion while measuring the patient's heat beats.

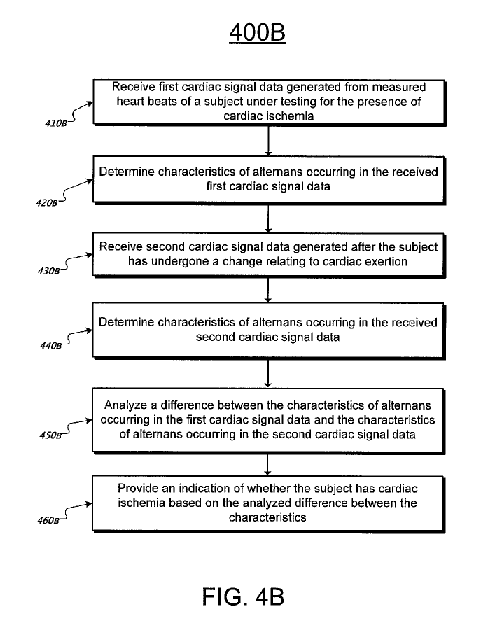

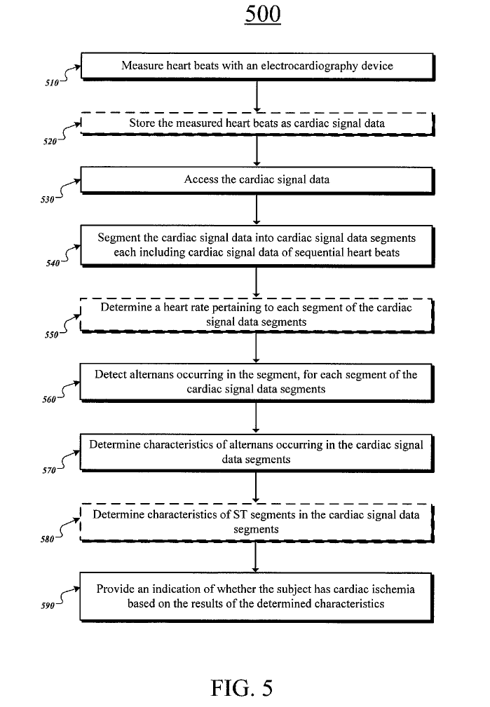

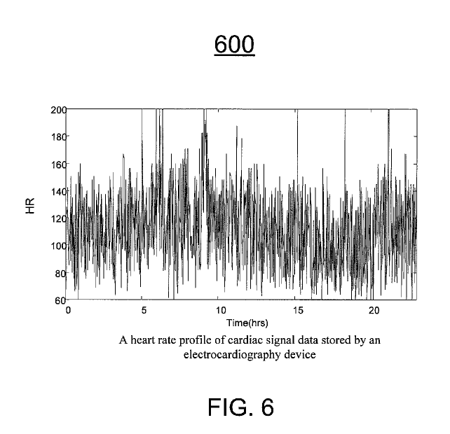

FIGS. 4A, 4B and 5 describe processes 400A, 400B and 500 to detect ischemia due to CAD. The processes 400A, 400B and 500 are described with respect to the features of FIGS. 2 and 3, though different electrocardiography devices and/or different features may be used. For example, the processes 400A, 400B and 500 can be conducted using an ambulatory or non-ambulatory electrocardiography device, with or without processing on a separate computer. Also, the below description of the process 500 refers to FIGS. 6-8, which are exemplary diagrams which can be representative of cardiac signal data analyzed during implementations of the process 500.

FIG. 4A illustrates a process 400A to detect ischemia due to CAD by analyzing the location of alternans within the waveform 100. Heart beats of a subject under testing for ischemia due to CAD, such as the patient 210, generate cardiac signals as voltages in the electrodes 220 which are measured by the electrocardiography device 300 to generate cardiac signal data.

The cardiac signal data generated from measured heart beats of the subject is received (410A). In one implementation, the processor 330 or a module thereof can access the cardiac signal data from volatile memory as it is generated by the electrocardiography device 300. In another implementation, the processor 330 stores the measured heart beats as cardiac signal data in non-volatile memory, and the stored data is later accessed by the electrocardiography device 300 or another device. Whether the receipt of the cardiac signal data (410A) is by the device generating the data or is by another device at a later time can be dependent on whether the device is a non-ambulatory electrocardiography device or an AED.

Alternans occurring in the received cardiac signal data is detected (420A). In particular, the received cardiac signal data can be analyzed for beat-to-beat variations that occur on an every-other-beat basis. The variations can be on the order of microvolts. Many implementations use spectral or analytic approaches to detect the occurrence of alternans in the cardiac signal data. These approaches are described in detail in U.S. Pat. No. 7,197,358, entitled “Identifying Infants at Risk for Sudden Infant Death Syndrome,” the contents of which are incorporated herein by reference. Further details of techniques to detect the occurrence of alternans are also described in reference to element 560 of the process 500 of FIG. 5. Detecting the occurrence of alternans (420A) can also include determining the presence or absence of alternans in the received cardiac signal data, the amount of alternans in the received cardiac signal data, or the duration of alternans in the received cardiac signal data.

Next, the location of detected alternans is analyzed within the waveform 100 (430A). The location of the alternans within the waveform can be characterized, for example, as occurring in the ST segment 140, the early or late part of the T-wave 150, or within the QRS complex 130.

Characteristics of the alternans or waveform 100 can also be determined. For example, the cardiac signal data can be analyzed to determine the average power of occurring alternans, the heart rate pertaining to the cardiac signal data with alternans, an onset heart rate of the alternans or a maximum heart rate below which alternans is not present (“maximum negative heart rate”), and the accompaniment of alternans with other characteristics of the waveform 100, such as depression or other abnormalities of the ST segment 140.

An indication of whether the subject is at risk for ischemia due to CAD is provided based on the location of the detected alternans within the waveform 100 (440A). The location of alternans within the waveform can be used to differentiate alternans resulting from ischemia due to CAD from alternans resulting from cardiomyopathy or other causes. Specifically, alternans occurring later in the T-wave 150 coincide with electrical recovery and can be indicative of non-ischemic causes. Alternans occurring earlier in the T-wave 150 or in the ST segment 140 or in the QRS complex 130 can relate to early repolarization of the heart muscles and can be indicative of ischemia due to CAD. In some implementations, the analyzed location of alternans within the waveform 100 is used to differentiate whether the patient has ischemia due to CAD or the alternans is due to cardiomyopathy or other causes. Specifically, alternans that occurs early in the T-wave 150 may indicate ischemia due to CAD, while alternans that occurs later in the T-wave 150 may indicate the presence of cardiomyopathy or other chronic conditions which predispose to SCD.

In some implementations, the indication is provided (440A) as a result of a determination which considers the location of the alternans within the waveform 100 as one of multiple factors. In particular, the additional characteristics described above can be taken into account in providing the indication. The indication may be a calculated result which qualitatively or quantitatively indicates the likelihood or severity of ischemia-related CAD. Specifically, a function taking into account the location of the alternans and other characteristics can be used to weight the variables according to importance or determined value to calculate a score. In one implementation, the score is a value between 0 and 10, with 0 indicating no risk or severity and 10 indicating a drastic risk or severity of ischemia-related CAD.

Multiple functions may be used in the comparison which are specifically tailored to identify different conditions or risks. For example, the indication can include a first score pertaining to ischemia due to CAD calculated using a first function and a second score pertaining to non-ischemic causes calculated using a second function. Early occurring T-wave alternans increase the first score and decrease the second score, while later occurring T-wave alternans decrease the first score and increase the second score.

FIG. 4B illustrates a process 400B to detect ischemia due to CAD by analyzing characteristics of alternans before and after cardiac exertion. Heart beats of the patient 210 under testing for ischemia due to CAD generate cardiac signals as voltages in the electrodes 220, which are measured by the electrocardiography device 300 to generate first cardiac signal data.

The first cardiac signal data generated from measured heart beats of the subject is received (410B). In one implementation, the processor 330 or a module thereof can access the first cardiac signal data from volatile memory as it is generated by the electrocardiography device 300. In another implementation, the processor 330 stores the measured heart beats as first cardiac signal data in non-volatile memory, and the stored data is later accessed by the electrocardiography device 300 or another device.

Characteristics of alternans occurring in the received first cardiac signal data are determined (420B). In one implementation, the characteristics of alternans consists of the presence or absence of alternans in the first cardiac signal data, the amount of alternans in the first cardiac signal data, or the duration of alternans in the first cardiac signal data.

In other implementations, the cardiac signal data is analyzed to determine additional characteristics. For example, a heart rate pertaining to portions of the first cardiac signal data can be determined, and based on the determined heart rate, the characteristics can include an onset heart rate of alternans or a maximum heart rate below which alternans is not present. Additional characteristics can include the magnitude of alternans or the accompaniment of alternans with other abnormalities of the waveform 100, such as depression of the ST segment 140 or other factors. Abnormalities of the waveform 100, such as depression of the ST segment 140, as discussed here are understood to be distinct form alternans in that these abnormalities persist over multiple beats while alternans represents a beat-to-beat pattern of variation.

The patient 210 is subjected to a change relating to cardiac exertion. The change can be part of an exercise stress test, and can include placing the patient 210 on a treadmill or increasing the speed of the treadmill. The change can also be administration of a pharmacological agent which dilates or activates the cardiovascular system of the patient 210. If the patient 210 is using an AED, the change can be a part of a daily routine such as walking, jogging, or climbing stairs. Heart beats of the patient 210 after the change in cardiac exertion further generate cardiac signals as voltages in the electrodes 220 which are measured by the electrocardiography device 300 to generate second cardiac signal data.

The second cardiac signal data generated after the subject has undergone a change relating to cardiac exertion is received (430B). Characteristics of alternans occurring in the received second cardiac signal data are determined (440B).

Thereafter, a difference between the characteristics of alternans occurring in the first cardiac signal data and the characteristics of alternans occurring in the second cardiac signal data is analyzed (450B). The analysis can include a qualitative or quantitative examination of differences between the characteristics. In particular, a difference can be calculated between the occurrence or characteristics of alternans (or other characteristics of the ECG waveform 100) prior to the change relating to cardiac exertion from that after the change relating to cardiac exertion. The difference can pertain to one or more of the factors described above as characteristics, such as, for example, the difference in whether alternans is present or the difference in the amount or duration of alternans, the onset heart rate or maximum negative heart rate of alternans, or the temporal location of alternans in the waveform.

An indication of whether the subject has ischemia due to CAD is provided based on the analyzed difference between the characteristics (460B). The difference can be used to differentiate alternans due to CAD from alternans due to non-ischemic causes such as cardiomyopathy or other abnormalities. Specifically, alternans occurring only after the change related to cardiac exertion (i.e., only within the second cardiac signal data) can be indicative of ischemia-related CAD, while alternans occurring regardless of the change (i.e., within both the first and second cardiac signal data) can be indicative of cardiomyopathy or a risk of SCD.

In some implementations, the indication is provided (460B) as a result of a determination which considers differences of multiple characteristics, such as, for example the alternans onset heart rate, the maximum heart below which alternans is not present, and the distribution of heart rates with alternans. Further information about the analysis and classification of measured alternans can be found at U.S. Pat. No. 6,453,191 entitled “Automated Interpretation of T-wave Alternans Results,” the contents of which are incorporated herein by reference. Multiple functions may be used in the comparison which are specifically tailored to identify different risks.

The indication may be a calculated numerical result which qualitatively indicates the likelihood or severity of ischemia-related CAD. Specifically, a function can be used to weigh the differences according to importance or determined value to calculate a score. Multiple functions may be used in the comparison which are specifically tailored to identify different conditions or risks. For example, the indication can include a first score pertaining to ischemia calculated using a first function and include a second score pertaining to risk of SCD calculated using a second function. Differences indicative of alternans characteristics occurring only after the change relating to cardiac exertion increase the first score and decrease the second score, while differences indicative of alternans characteristics occurring regardless of the change relating to cardiac exertion decrease the first score and increase the second score.

In some implementations, the subject is monitored only after the change related to cardiac exertion. The alternans characteristics are compared to a known expected alternans characteristic or lack thereof (e.g., what may be considered “normal” cardiac function). Also, the process 400B can be implemented in a different order. For example, element 420B can occur after element 430B. Specifically, if the electrocardiography device 300 used is an AED, the first and cardiac signal data may be generated and stored as cardiac signal data or segmented cardiac signal data (discussed below) on the AED. Thereafter, a separate computer can access, retrieve, and further process the first and second cardiac signal data.

FIG. 5 illustrates a process 500 to detect ischemia due to CAD using an electrocardiography device 300. The process 500 can be particularly useful where data is recorded by an AED for later processing by a separate device. Nevertheless, the process 500 can be carried out with data generated by the electrocardiography device 300 as the data is generated. The description of the process 500 can be applicable to the processes 400A and 400B and vice versa.

Initially, a subject's heart beats are measured with the electrocardiography device 300 (510). Specifically, the electrocardiography device 300 amplifies and digitizes the voltages from the electrodes 220 to enable digital signal processing by the processor 330 of the electrocardiography device 300 to generate cardiac signal data. The cardiac signal data can be generated from measured heart beats of the patient 210 prior to, during, or after a change pertaining to cardiac exertion. In some implementations, the measured heart beats are stored as cardiac signal data (520) in the data storage 340. For example, many AEDs store the cardiac signal data in transferable memory (e.g., a flash drive) to enable the data to be further processed elsewhere. The electrocardiography device 300 may store the cardiac signal data along with one or more data headers indicating the nature of the data, such as indicating the data is before, during, or after the change pertaining to cardiac exertion based on input received from the user input control 350.

The cardiac signal data generated from heart beats measured with the electrocardiography device 300 can be accessed by the electrocardiography device 300 or a separate device (530). By using the separate device in further processing, the electrocardiography device 300 can be of minimal size and complexity. Nevertheless, a more advanced electrocardiography device 300 with additional processing power and programming can implement the further processing discussed below without the use of a separate device.

FIG. 6 is a diagram 600 of an example of a heart rate profile of the cardiac signal data stored by the electrocardiography device 300. The diagram 600 shows the cardiac signal data produced from the cardiac signals measured by the electrocardiography device 300 during a 24 hour period. The cardiac signal data is presented as heart rate as a function of time. The diagram 600 illustrates a challenge of using the cardiac signal data produced by the electrocardiography device 300 to detect alternans and its characteristics. As described above, alternans can represent an every other beat pattern of variation in portions of the waveform 100 of a measured cardiac signal. For example, T-wave alternans can be microvolt-level variations in the amplitude of the T-wave from one beat to the next, generally observed during heart rates of 100 to 120 BPM for patients with ischemia-related CAD. Optimally, to detect this alternans, the cardiac signal data is obtained at heart rates between 100 and 120 BPM, and is maintained at that level long enough to repeatedly analyze the beat-to-beat variation. However, the cardiac signal data of the diagram 600 is not consistently at the desired heart rate and is not maintained at a given level. Although there are instances where the heart rate is between 100 and 120 BPM, these instances are scattered and not ideal for the detection of alternans.

The cardiac signal data stored by the electrocardiography device 300 is processed to convert the scattered cardiac data of the diagram 600 into more useful data, such as segments organized by associated heart rates. Simply sorting the cardiac signal data by heart rate for each beat can foreclose the detection of variations between consecutive beats. Therefore, to preserve the beat-to-beat nature of the cardiac signal data, the processing can involve segmenting data into groups of adjacent beats, determining features of the segments, and sorting the segments by one or more features prior to processing to determine and compare alternans characteristics.

The cardiac signal data is segmented into cardiac signal data segments (540). Each segment of the cardiac signal data segments includes data associated with multiple consecutive heartbeats. In one implementation, the segments are of 128 beats, but other segment sizes can be used. The segments can overlap beats so as to ensure the temporal relationship of beats is not lost. For example, the first 248 beats of cardiac signal data can be segmented into a first segment of beats 1 to 128 and a second segment of beats 120 to 248, leaving beats 120-128 included in both segments. Therefore, beat-to-beat variations in beats 120-128 can be compared to beats occurring just prior to beats 120-128 as well as to beats occurring just after beats 120-128.

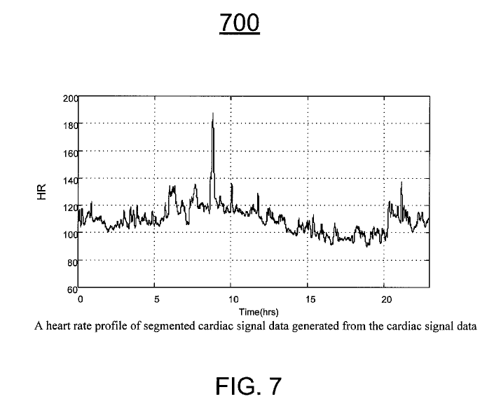

In some implementations, a heart rate pertaining to each segment of the cardiac signal data segment is determined (550). In particular, a heart rate is separately calculated for each segment of the cardiac signal data. The heart rate can be based on a simple averaging of the duration of each of the heart beats of a segment. FIG. 7 is a diagram 700 of an example of a heart rate profile of segmented cardiac signal data generated from the cardiac signal data of FIG. 6. The diagram 700 shows the segmented cardiac signal data as heart rate as a function of time. Notably, the heart rate of the segmented cardiac signal data in the diagram 700 fluctuates less dramatically than the heart rate of the cardiac signal data of individual heart beats as shown in the diagram 600.

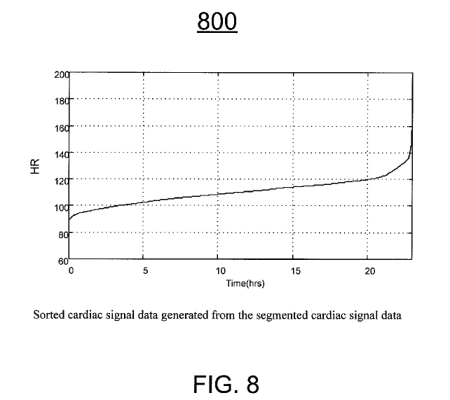

In some implementations, the cardiac signal data segments are sorted into an order from the lowest determined heart rate to the highest determined heart rate. FIG. 8 is a diagram 800 of an example of sorted cardiac signal data segments generated from the segmented cardiac signal data of FIG. 7. The diagram 800 shows the distribution of heart rates for the segments after the segments have been ordered from the lowest determined heart rate to the highest determined heart rate. Although this exemplary distribution shows that the majority of the cardiac signal data segments fall within the desired heart rate of 100 to 120 BPM, other distributions from other patients can have only a small fraction of the cardiac signal data segments within the desired heart rate.

Alternans is detected for each segment of the cardiac signal data segments (or for each segment of the cardiac signal data segments corresponding to suitable heart rates) (560). In particular, each of the cardiac signal data segments can be separately processed to detect alternans. Therefore, each of the cardiac signal data segments can have a unique determination of the presence of alternans. Many implementations use spectral or analytic approaches to detect the occurrence of alternans in the cardiac signal data. In the example above, where the first 248 beats of cardiac signal data are segmented into a first segment of beats 1 to 128 and a second segment of beats 120 to 248, the first segment is analyzed using the spectral or analytical approach to determine a first result, and the second segment is then analyzed using the spectral or analytical approach to determine a second result.

The analysis of the cardiac signal data segments can also include processing dependent upon the determined heart rate or other characteristics of the cardiac signal data segments. In some implementations, cardiac signal data segments outside of a given range may be discarded or separately considered. Also, processing can be conducted differently based upon the determined heart rate.

Turning to the spectral approach, this approach uses measurements from time synchronized points of consecutive waveforms. For a portion of the cardiac signal data segment, a time series is created by measuring, for each of the heart beats, the ECG voltage at a fixed time offset with relation to the QRS complex 130 of the waveform. This process is repeated to create a set of time series corresponding to a set of different offsets each falling within a specific section (e.g. the ST segment or the T-wave) of the waveform. A frequency spectrum is then generated for each time series, and the spectra are averaged to form a composite alternans spectrum corresponding to the selected section of the waveform.

Since one sample per beat is obtained for each point in each individual time series, the spectral value at the Nyquist frequency, i.e. 0.5 cycles per beat, indicates the level of beat-to-beat alternation in the selected section of the waveform. The alternans power is calculated from the composite alternans spectrum and statistically compared to the noise power to discriminate the alternating beat-to-beat variation in the waveform due to abnormal electrical activity of the heart from the random variation due to background noise. Alternans may be considered to be significant if the alternans exceed noise by a threshold amount, such as at least three times the standard deviation of the noise in a given noise reference band.

One example of how processing can be conducted differently based upon the determined heart rate is using a different threshold for determining whether alternans are significant for cardiac signal data segments of different heart rate ranges. For example, alternans of cardiac signal data segments with determined heart rates below 100 BPM may be considered significant if the alternans are at least double the standard deviation of the noise in the noise reference band, whereas alternans of cardiac signal data segments with determined heart rates above 100 BPM may be considered significant if the alternans are at least triple the standard deviation of the noise in the noise reference band.

Turning to the analytic approach, this approach can be used to minimize the presence of noise or artifacts. First, a segment of the cardiac signal data segments is low-pass filtered. In one implementation, the low pass filter is a 5^{th} order Butterworth filter with a zero phase configuration. The segment is transferred to the frequency domain using a fast Fourier transform (FFT). In the frequency domain, the portions of the frequency spectrum corresponding to negative frequencies are removed and all positive, non-zero components of the frequency spectrum are doubled to compensate. An inverse fast Fourier transform (IFFT) is then performed on the modified frequency spectrum to produce an analytical data segment in the time domain. Next, the analytical data segment is referenced to an analytical version of Wilson's central terminal (WCT), which is an ECG reference value. The analytical version of WCT is generated from the standard WCT using the procedures described in U.S. Pat. No. 7,197,358, title “Identifying infants at risk for sudden infant death syndrome” and U.S. Pat. No. 5,704,365, titled “Using Related Signals to Reduce ECG Noise,” the contents of both are incorporated herein by reference. The analytical data segment is referenced to the analytical version of WCT by determining the difference between the two. The referenced analytical data segment then is processed.

If the data from the electrocardiography device 300 includes signals used to determine and adjust for noise (e.g., signals related to respiration and impedance), the time series can be processed to reduce noise, such as that resulting from baseline wander. Techniques for processing the time series are described in more detail in U.S. Pat. No. 5,704,365, titled “Using Related Signals to Reduce ECG Noise,” the contents of which are incorporated herein by reference.

Next, characteristics of alternans occurring in the cardiac signal data segments are determined (570). In some implementations, the characteristics of alternans include the presence or absence of alternans within the waveform 100 in the cardiac signal data segments, the amount of or duration of alternans within the waveform 100 in the cardiac signal data segments, or the location of alternans within the waveform 100 in the cardiac signal data segments (as described above). For example, the determined characteristics can consist of a determination of the extent of the presence of alternans within the cardiac signal data segments. Also, the determined characteristics can include the extent of the presence of alternans (e.g., the number of segments to which alternans occur, the average power of occurring alternans, or a function taking into account the amount of alternans presence and their power).

In other implementations, the cardiac signal data segments are further analyzed to determine additional features as part of the determined characteristics. In particular, the occurrences of alternans in the cardiac signal data segments can be compared to the context of the occurrences to determine further information. The context of the occurrence can include the heart rate pertaining to the cardiac data segment, the temporal position of a cardiac signal data segment with alternans present relative to other cardiac signal data segments, the consecutive duration of cardiac signal data segments with alternans, the time or heart rate of the cardiac signal data segment with alternans present, or other considerations. For example, based on the determined heart rate of the cardiac signal data segments, the determined characteristics can include an onset heart rate or a maximum negative heart rate of alternans, for a particular segment or for all segments of the cardiac signal data segments.

In some implementations, characteristics of ST segments 140 in the cardiac signal data segments are also determined (580). The characteristics of the ST segments 140 can include, for example, the duration, magnitude, slope, or concavity. The characteristics can be relevant as they may differ in healthy patients as compared to those with CAD. In a healthy patient, the ST segment 140 slopes slightly upward. A downward sloping or overly flat ST segment 140 can indicate the existence of ischemia due to CAD. It is understood that the characteristics of the ST segment described in this paragraph are distinct from alternans in that these characteristics persist over multiple beats whereas alternans represents a beat-to-beat pattern of variation.

In some implementations, the accessed cardiac signal data includes first and second cardiac signal data produced from measured heat beats occurring prior to and after cardiac exertion, similar to the process 400B of FIG. 4B. The segmenting and determination of characteristics can be conducted separately upon the first and second cardiac signal data. One or more of the determined characteristics of the first and second cardiac signal data segments can be analyzed to determine a difference between the characteristics occurring in the first cardiac signal data segments and the characteristics occurring in the second cardiac signal data segments (not shown). For example, the comparison can include a determination that alternans occurred in the second cardiac signal data segments twice as often as in the first cardiac signal data segments.

An indication of whether the subject has ischemia due to CAD is provided based on the results of the determined characteristics (590). The indication can be generated through consideration of the location of alternans within the waveform 100 or the differences between characteristics before, during, or after cardiac exertion. The indication may be a calculated result which qualitatively or quantitatively indicates the likelihood or severity of ischemia-related CAD or other diseases or risks. One or more functions may be used which are specifically tailored to identify the different conditions or risks. For example, the indication can include a first score pertaining to ischemia-related CAD calculated using a first function and a second score pertaining to SCD calculated using a second function.

As noted above, the processes 400A-500 can be carried out using an AED to measure cardiac signals and store cardiac signal data and using a separate computer to conduct further processing. More advanced AEDs can be programmed to themselves carry out the processing of the processes 400A-500. For example, in some implementations, an AED itself segments the data, detects alternans or characteristics, and/or provides the indication using the processor of the AED concurrent with the measuring of cardiac signal data. In these implementations, the AED may store and access the cardiac signal data, generated cardiac signal data segments, determined characteristics, or any other information discussed above in and from volatile memory along with or instead of non-volatile memory to enable further processing to be carried out concurrently with measurement rather than after measurement. For example, in some implementations, segmented first and second cardiac data is stored in the AED's data storage and is later accessed by another device.

FIG. 9 is a schematic of an example of a computer system 900 configured to carry out the processes 400A-500 of FIGS. 4A-5. The description of the computer system 900 can also apply to the hardware and functioning of the electrocardiography device 300 or an AED.

The computer system 900 includes a processor 910, memory 920, and an input/output device 940. The components 910, 920, and 940 are interconnected using a system bus 950. The processor 910 is capable of processing instructions for execution within the computer system 900. In one implementation, the processor 910 is a single-threaded processor. In another implementation, the processor 910 is a multi-threaded processor. The processor 910 is capable of processing instructions stored in the memory 920 to display graphical information for a user interface on the input/output device 940.

The memory 920 stores information within the computer system 900 and includes volatile memory 930 and non-volatile memory 935 and can be a computer-readable medium tangibly embodying instructions. The volatile memory 930 can include random access memory (RAM) and semiconductor memory devices (e.g., flip-flops or registers). The non-volatile memory 935 is capable of providing mass storage for the computer system 900. In various implementations, the non-volatile memory 935 can be a floppy disk device, a hard disk device, an optical disk device, or a tape device. Also, the non-volatile memory 935 can include, or be operatively coupled to communicate with, one or more mass storage devices for storing data files; such devices include magnetic disks, such as internal hard disks and removable disks, magneto-optical disks, optical disks, EPROM, EEPROM, flash memory devices, and CD-ROM, DVD-ROM, or Blu-ray™ disks.

The input/output device 940 provides input/output operations for the computer system 900. In one implementation, the input/output device 940 includes a keyboard and/or pointing device. In another implementation, the input/output device 940 includes a display unit for displaying graphical user interfaces. The input/output device 940 can include communications input/output operations. For example, the input/output device 940 can include a port for connection flash drives or other memory devices through a universal serial bus or other connection. Also, the input/output device 940 can include an Ethernet port for communication with other devices.

The features and processing described above can be implemented in a computer program product tangibly embodied in an information carrier, e.g., in a computer-readable medium encoded with a computer program product or in a machine-readable storage device for execution by a programmable processor; and features of the methods may be performed by a programmable processor executing a program of instructions to perform functions of the described implementations by operating on input data and generating output.

The described features and processing may be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that may be used, directly or indirectly, in a computer to perform a certain activity or bring about a certain result. A computer program may be written in any form of programming language, including compiled or interpreted languages, and it may be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. The processor and the memory may be supplemented by, or incorporated in, ASICs (application-specific integrated circuits).

To provide for interaction with a user, the features may be implemented on a computer having a display device such as a CRT (cathode ray tube) or LCD (liquid crystal display) monitor for displaying information to the user and a keyboard and a pointing device such as a mouse or a trackball by which the user may provide input to the computer.

The components of the system may be connected by any form or medium of digital data communication such as a communication network. Examples of communication networks include, e.g., a LAN, a WAN, and the computers and networks forming the Internet.

A number of implementations have been described. Nevertheless, it will be understood that various modifications may be made without departing from the spirit and scope of the claims. For example, the flow diagrams depicted in the figures do not require the particular order shown, or sequential order, to achieve desirable results. In addition, other features may be provided, or features may be eliminated, from the described block diagrams, and other components may be added to, or removed from, the described devices. Accordingly, other implementations are within the scope of the following claims.

What is claimed is:

1. A method for detecting cardiac ischemia, the method comprising:
receiving one or more electrocardiographic signals from a subject;
detecting, using at least one processor, the occurrence of alternans in the electrocardiographic signals;
determining one or more characteristics of detected alternans; and
analyzing the determined characteristics of the detected alternans to determine whether cardiac ischemia is present.

2. The method of claim 1 wherein:
determining the characteristics of the detected alternans includes determining the location of detected alternans within an electrocardiogram waveform; and
analyzing the determined characteristics of the detected alternans includes analyzing the determined location of the detected alternans to determine whether the cardiac ischemia is present.

3. The method of claim 1 wherein:
determining the characteristics of the detected alternans includes evaluating a relationship of the detected occurrence of the alternans to cardiac stress; and
analyzing the determined characteristics of the detected alternans includes analyzing the evaluated relationship to provide an indication of whether the subject has cardiac ischemia.

4. The method of claim 1 wherein receiving one or more electrocardiographic signals from the subject includes receiving one or more electrocardiographic signals from the subject while the subject is undergoing a stress test.

5. The method of claim 4 wherein the stress is exercise stress, pharmacological stress, or stress induced by electrically pacing the heart.

6. The method of claim 1 wherein determining the characteristics of the detected alternans includes determining an onset heart rate of alternans or a maximum heart rate below which alternans is not present.

7. The method of claim 1 further comprising:
determining the occurrence, in the electrocardiographic signals, of abnormalities that persist over multiple beats and are indicative of cardiac ischemia; and
providing an indication of whether the subject has cardiac ischemia based on the determined characteristics of the alternans and the determination of the occurrence of abnormalities that persist over multiple beats and are indicative of cardiac ischemia.

8. The method of claim 7 wherein the abnormalities are alterations in the ST segment.

9. The method of claim 8 wherein the alterations in the ST segment are depression or elevation of the ST segment or a change in the slope of the ST segment.

10. The method of claim 1 wherein the electrocardiographic signals are received using an ambulatory electrocardiography device.

11. The method of claim 1 wherein the alternans is detected using a spectral method of analysis.

12. The method of claim 1 wherein the alternans is detected using an analytic method of analysis.

13. The method of claim 1 further comprising:
obtaining non-electrocardiographic measures indicative of the presence of cardiac ischemia; and
providing an indication of whether the subject has cardiac ischemia based on the obtained non-electrocardiographic measures indicative of the presence of cardiac ischemia.

14. The method of claim 13 wherein the non-electrocardiographic measures are measured using echocardiographic imaging of a heart or by characterizing the uptake of radionuclides by the heart.

15. The method of claim 1 wherein determining one or more characteristics of the detected alternans includes determining a power or magnitude of alternans.

16. The method of claim 1 wherein detecting the occurrence of alternans in the electrocardiographic signals includes detecting the occurrence of T-wave alternans occurring in the electrocardiographic signals.

17. The method of claim 1 wherein detecting the occurrence of alternans in the electrocardiographic signals includes detecting the occurrence of QRS complex alternans occurring in the electrocardiographic signals.

18. The method of claim 1 wherein detecting the occurrence of alternans in the electrocardiographic signals includes detecting the occurrence of ST segment alternans occurring in the electrocardiographic signals.

19. The method of claim 1 further comprising:
generating cardiac signal data from the electrocardiographic signals; and
segmenting the cardiac signal data into cardiac signal data segments which include cardiac signal data of sequential heart beats.

20. The method of claim 19 wherein at least one cardiac signal data segment partially overlaps the cardiac signal data of at least one other cardiac signal data segment.

21. The method of claim 19 further comprising sorting the cardiac signal data segments.

22. A computer-readable medium encoded with a computer program comprising instructions that, when executed, operate to cause one or more computers to perform operations, the operations comprising:
receiving cardiac signal data generated from measured heart beats of a subject;
detecting the occurrence of alternans in the cardiac signal data;
determining one or more characteristics of detected alternans in the cardiac signal data; and
providing an indication related to cardiac ischemia based on the determined characteristics of detected alternans.

23. The medium of claim 22 wherein:
determining the characteristics of the detected alternans includes determining the location of detected alternans within an electrocardiogram waveform; and
providing the indication related to cardiac ischemia based on the detected occurrence of alternans includes providing the indication related to cardiac ischemia based on the determined location of detected alternans.

24. The medium of claim 22 wherein detecting the occurrence of alternans in the cardiac signal data includes detecting the occurrence of T-wave alternans in the cardiac signal data.

25. The medium of claim 22 wherein detecting the occurrence of alternans in the cardiac signal data includes detecting the occurrence of QRS complex alternans in the cardiac signal data.

26. The medium of claim 24 wherein detecting the occurrence of alternans in the cardiac signal data includes detecting the occurrence of ST segment alternans in the cardiac signal data.

27. The medium of claim 22 wherein receiving the cardiac signal data includes accessing stored cardiac signal data from a non-volatile data storage, wherein the cardiac signal data was stored by an ambulatory electrocardiography device.

28. The medium of claim 22 wherein receiving the cardiac signal data includes accessing cardiac signal data from volatile memory which has not been stored in a non-volatile data storage.

29. The medium of claim 22 wherein determining the characteristics of the detected alternans includes determining an onset heart rate of alternans or a maximum heart rate below which alternans is not present in the cardiac signal data.

30. The medium of claim 22 further comprising:
determining the occurrence, in the cardiac signal data, of abnormalities that persist over multiple beats and are indicative of cardiac ischemia,
wherein providing the indication related to cardiac ischemia based on the determined characteristics of detected alternans includes providing the indication of whether the subject has cardiac ischemia based on the determined characteristics of detected alternans and the determination of the occurrence of abnormalities that persist over multiple beats and are indicative of cardiac ischemia.

31. The medium of claim 30 wherein the abnormalities are alterations in the ST segment.

32. The medium of claim 31 wherein the alterations in the ST segment are depression or elevation of the ST segment or a change in the slope of the ST segment.

33. A system comprising:
sensors configured to measure electrical activity of heart beats;
an amplifier configured to amplify the electrical activity;
an analog to digital converter configured to convert the electrical activity to cardiac signal data; and
a processor configured to:
receive the cardiac signal data generated from measured heart beats of a subject,
detect the occurrence of alternans in the cardiac signal data,
determine one or more characteristics of detected alternans in the cardiac signal data, and
provide an indication related to cardiac ischemia based on the determined characteristics of detected alternans.

34. The system of claim 33 wherein:
to determine the characteristics of the detected alternans, the processor is configured to determine the location of detected alternans within an electrocardiogram waveform; and
to provide the indication related to cardiac ischemia based on the detected occurrence of alternans, the processor is configured to provide the indication related to cardiac ischemia based on the determined location of detected alternans.

35. The system of claim 33 wherein, to detect, the occurrence of alternans in the cardiac signal data, the processor is configured to detect the occurrence of T-wave alternans in the cardiac signal data.

36. The system of claim 33 wherein, to detect, the occurrence of alternans in the cardiac signal data, the processor is configured to detect, the occurrence of QRS complex alternans in the cardiac signal data.

37. The system of claim 33 wherein, to detect, the occurrence of alternans in the cardiac signal data, the processor is configured to detect, the occurrence of ST segment alternans in the cardiac signal data.

38. The system of claim 33 wherein, to determine the characteristics of the detected alternans, the processor is configured to determine an onset heart rate of alternans or a maximum heart rate below which alternans is not present in the cardiac signal data.

39. The system of claim 33 wherein:
the processor is configured to determine the occurrence, in the cardiac signal data, of abnormalities that persist over multiple beats and are indicative of cardiac ischemia; and
to provide the indication related to cardiac ischemia based on the determined characteristics of detected alternans, the processor is configured to provide the indication of whether the subject has cardiac ischemia based on the determined characteristics of detected alternans and the determination of the occurrence of abnormalities that persist over multiple beats and are indicative of cardiac ischemia.

40. A method for detecting cardiac ischemia, the method comprising:
receiving first cardiac signal data generated from measured heart beats of a subject;
determining characteristics of alternans occurring in the first cardiac signal data;
receiving second cardiac signal data generated from measured heart beats of the subject after the subject has undergone a change relating to cardiac stress;
determining characteristics of alternans occurring in the second cardiac signal data;
analyzing a difference between the characteristics of alternans occurring in the first cardiac signal data and the characteristics of alternans occurring in the second cardiac signal data; and
providing an indication related to cardiac ischemia based on the analyzed difference between the characteristics of alternans.

41. The method of claim 40 wherein:
determining characteristics of alternans occurring in the first cardiac signal data includes determining the location of alternans occurring in the first cardiac signal data;
determining characteristics of alternans occurring in the second cardiac signal data includes determining the location of alternans occurring in the second cardiac signal data;
analyzing a difference between the characteristics includes analyzing a difference between the location of alternans occurring in the first cardiac signal data and the location of alternans occurring in the second cardiac signal data; and
providing the indication related to cardiac ischemia based on the analyzed difference between the characteristics of alternans includes providing the indication related to cardiac ischemia based on the analyzed difference between the location of alternans occurring in the first cardiac signal data and the location of alternans occurring in the second cardiac signal data.

42. The method of claim 40 wherein:
determining characteristics of alternans occurring in the first cardiac signal data includes determining a power or magnitude of alternans in the first cardiac signal data;
determining characteristics of alternans occurring in the second cardiac signal data includes determining a power or magnitude of alternans in the second cardiac signal data;
analyzing the difference includes determining the difference between the power or magnitude of alternans in the first cardiac signal data and the power or magnitude of alternans in the second cardiac signal data; and
providing the indication includes assessing whether the difference between the power or magnitude of alternans is indicative of cardiac ischemia.

43. The method of claim 40 wherein:
determining characteristics of alternans occurring in the first cardiac signal data includes determining an onset heart rate of or a maximum heart rate without alternans in the first cardiac signal data;
determining characteristics of alternans occurring in the second cardiac signal data includes determining an onset heart rate of or a maximum heart rate without alternans in the second cardiac signal data;
analyzing the difference includes determining the difference between the onset heart rate of or the maximum heart rate without alternans in the first cardiac signal data and the onset heart rate of or the maximum heart rate without alternans in the second cardiac signal data; and
providing the indication includes assessing whether the difference between the onset heart rate of or the maximum heart rate without alternans is indicative of cardiac ischemia.

44. The method of claim 40 wherein receiving the first and second cardiac signal data includes accessing stored cardiac signal data from a non-volatile data storage, wherein the cardiac signal data was stored by an ambulatory electrocardiography device.

45. The method of claim 40 further comprising:
determining the occurrence, in the first cardiac signal data, of abnormalities that persist over multiple beats and are indicative of cardiac ischemia;
determining the occurrence, in the second cardiac signal data, of abnormalities that persist over multiple beats and are indicative of cardiac ischemia; and
analyzing a difference between the occurrence of abnormalities in the first cardiac signal data and the occurrence of abnormalities in the second cardiac signal data,
wherein providing the indication related to cardiac ischemia based on the analyzed difference between the characteristics of alternans includes providing the indication related to cardiac ischemia based on the analyzed difference between the characteristics of alternans and based on the analyzed difference between the occurrence of abnormalities.

46. The method of claim 40 further comprising:
segmenting the first cardiac signal data into first cardiac signal data segments, each first cardiac signal data segment including cardiac signal data of sequential heart beats; and
segmenting the second cardiac signal data into second cardiac signal data segments, each second cardiac signal data segment including cardiac signal data of sequential heart beats, wherein
determining characteristics of alternans occurring in the first cardiac signal data includes determining characteristics of alternans occurring in the first cardiac signal data segments,
determining characteristics of alternans occurring in the second cardiac signal data includes determining characteristics of alternans occurring in the second cardiac signal data segments, and
analyzing the difference includes analyzing the difference between the characteristics of alternans occurring in the first cardiac signal data segments and the characteristics of alternans occurring in the second cardiac signal data segments.

47. The method of claim 46 wherein the first and second cardiac signal data is segmented such that the sequential order of the heart beats as measured by sensors is maintained within the first and second cardiac signal data segments.

48. The method of claim 46 wherein the first and second cardiac signal data is segmented such that the cardiac signal data in at least one cardiac signal data segment partially overlaps the cardiac signal data of another cardiac signal data segment.
